(19) United States
(12) Patent Application Publication
(10) Pub. No.: US 2010/0114284 A1
(43) Pub. Date: May 6, 2010

(54) IMPLANTABLE LINE

(76) Inventor: Thomas DOERR, Berlin (DE)

Correspondence Address: DALINA PATENT GROUP, 7910 IVANHOE AVE. #325, LA JOLLA, CA, 92037, US

(21) Appl. No.: 12/605,976

(22) Filed: Oct. 26, 2009

(30) Foreign Application Priority Data
Nov. 6, 2008 (DE) 10 2008 043 513.9

(51) Int. Cl. A61N 1/05; A61M 25/01

(52) U.S. Cl. 607/120; 604/523; 604/531

(57) ABSTRACT

An implantable line for implantation in the left ventricle or left atrium of the heart with perforation of the atrial or ventricular septum, having an elongated flexible line body and an electrode and/or a sensor and/or a medication-dispensing device in a distal segment of the line body, i.e., in the left atrium or the left ventricle in the use state of the, such that the part of the line body situated in the blood vessel in or on the heart in the use state is at least partially provided with a surface coating or a surface structure that promotes ingrowth with endogenous tissue.

This application takes priority from German Patent Application DE 10 2008 043 513.9, filed 6 Nov. 2008, the specification of which is hereby incorporated herein by reference.

### BACKGROUND OF THE INVENTION

1. Field of the Invention

One or more embodiments of the invention relates to an implantable line, in particular an electrode line, a sensor line or a medication supply line, which is provided in a blood vessel in or on the heart, in particular for implantation in the left ventricle or the left atrium of the heart with perforation of the corresponding septum.

2. Description of the Related Art

Implantation of an electrode in a left-ventricular vein via the coronary sinus is currently considered to be the state of art for left-ventricular stimulation and detection.

The literature contains numerous case reports of transseptal implantation of left-ventricular stimulation electrodes for resynchronization therapy. These implantation techniques have always been conducted with the assistance of the existing catheters, guide wires and normal electrodes, i.e., right-ventricular electrodes or even inadvertently. As access to the left ventricle, puncture of either the atrial septum or the ventricular septum has been described, e.g., by van Gelder B M, Scheffer M G, Meijer A, et al. Transseptal endocardial left-ventricular pacing: An alternative technique for coronary sinus lead placement in cardiac resynchronization therapy. [Journal Article] Heart Rhythm 2007 April; 4(4):454-60.

Additional applications of permanently implanted electrodes include the use of sensors in the left atrium or ventricle or administration of medication on the left side by means of a permanently implanted catheter.

Implantation of a left-ventricular/left-atrial electrode is not being performed at the present time because there are considerable objections concerning the risk of development of thrombi with this electrode and the associated risk of a stroke. Although the left-ventricular/left-atrial flow rates speak against development of a thrombus, flow artifacts at a blood flow rate of <0.4 m/sec may be caused by the electrode. If these conditions persist for a long time, these areas may begin to form a thrombus.

The same is also true of the aforementioned applications of “hardwired” sensors or catheters for administering medication to the left side of the heart.

### BRIEF SUMMARY OF INVENTION

To eliminate the disadvantages of the related art, the object of embodiments of the invention is to design a catheter and/or a probe for permanent left-ventricular/left-atrial implantation, such that the risk of development of a thrombus in the area of the left ventricle or atrium is significantly reduced and thus it is possible to use a permanently implanted left-ventricular or left-atrial probe.

This object is achieved by a line having the features of at least the independent claim(s). Expedient further embodiments of the inventive idea are the subject of the dependent claims.

One or more embodiments of the invention is based on the idea that the risk of development of a thrombus is greatly reduced in the case of a probe completely enclosed in endogenous tissue.

A line and/or a catheter for permanent implantation in a blood vessel in or on the heart for detecting or delivering electric signals or as a sensor or for intracardiac administration of medication comprises according to embodiments of the invention a flexible elongated probe body having at least one electric pole and/or at least one sensor and/or at least one medication applicator into the blood vessel in or on the heart and preferably a possibility of fixation (by screw, hook, . . . ) of the probe in a section of the wall of the blood vessel in or on the heart, such that the part of the electrode body, which extends into the blood vessel in the use state is provided with a surface coating or a surface structure promoting ingrowth of the probe part with endogenous tissue.

A blood vessel in or on the heart here refers to coronary vessels, such as veins of the coronary sinus, a pulmonary vein or the aorta. More preferably, however, this refers to the chambers of the heart, such as the right and left ventricles or the right and left atria. In particular, the catheter or the line should be implanted permanently in the left atrium or the left ventricle, namely with perforation of the atrial or ventricular septum, as is known from DE 10 2008 040 304.0, for example. Perforation of the ventricular myocardium, e.g., starting from a coronary vessel or through the epicardium from the outside, is also possible.

The surface coating or surface structure which promotes ingrowth is preferably provided in the area of the line body situated near a wall of the left ventricle or left atrium during use.

One advantage of the inventive approach is the reduction in the thrombotic risk of a probe implanted permanently in a blood vessel in or on the heart. This makes it possible to use such a probe at all.

Advantages of the left-ventricular stimulation include:
more favorable stimulation site physiologically,
better sensing signals based on the larger muscle mass,
better conditions for attachment of the probe and a lower risk of perforation due to the greater wall thickness,
better possibilities of hemodynamic optimization by stimulation,
disadvantages of RV stimulation are largely eliminated.

Among other things, embodiments of the invention make it possible to insert the new type of electrodes and thus a new stimulation concept in the CRM field. In addition, application of sensors (pressure) or medication catheters for permanent left-ventricular/left-atrial implantation is made possible.

In one embodiment, the electrode has a “stent-like” structure for stimulation of the enclosure with endogenous tissue.

In one embodiment, the surface structure or surface coating is made of an absorbable material, which is completely absorbed by endogenous tissue after conclusion of the enclosure with endogenous tissue (e.g., magnesium, magnesium alloy such as WE 43 or a bioabsorbable polymer such as poly-L-lactide).

In the case of a bioabsorbable polymer as the surface structure or surface coating, the polymer contains an active ingredient for promoting ingrowth or, additionally or alternatively, an active ingredient for inhibiting platelet aggregation until complete enclosure with endogenous tissue.

In another embodiment, the surface coating consists of silicon carbide (SiC).

In one embodiment, the surface coating or surface structure is applied to a carrier material, embedded in a flexible line body, the carrier material preferably being a continuous layer situated between the line body on the surface coating or surface structure. In one embodiment, the carrier material contains active ingredients for promoting ingrowth in a depot storage form.

In another embodiment, the carrier material alternatively or additionally contains suitable active ingredients for inhibiting platelet aggregation until completely enclosed in endogenous tissue.

In one embodiment, the probe part, which is in a blood vessel in or on the heart, is preshaped such that the probe is largely in contact with the myocardium (contact with the substrate to promote enclosure with endogenous tissue). The electrode in the aforementioned area is logically preshaped (J, U or S shape), so that, after being implanted, it is mostly in contact with the wall and/or the septum of the left ventricle. Complete enclosure with endogenous tissue is thus stimulated. The position of an area provided with a surface coating is preferably coordinated with the prefabricated curvature of the line body, such that a section of the line body carries the surface coating or the surface structure, which is in contact with the wall of the blood vessel in or on the heart in the use state.

In one embodiment, the surface coating consists of mechanical microstructures with a structural size between 1 and 500 μm, preferably between 5 and 200 μm and more preferably between 10 and 50 μm (ideally ˜16 μm).

In another embodiment, the surface coating consists of mechanical macrostructures (e.g., a helical structure, a mesh structure, a honeycomb structure or the like).

The line may preferably comprise multiple sensing and/or stimulation and/or shock electrodes, such that preferably at least a portion of the electrode is provided with a surface coating or surface structure, in particular a stent-like or mesh-like or helical structure that promotes ingrowth.

### BRIEF DESCRIPTION OF THE DRAWINGS

Advantages and expediencies of embodiments of the invention are also derived from the following description of exemplary embodiments and aspects of embodiments of the invention on the basis of the figures, in which:

FIG. 1 shows an overall view of an implanted medical device arrangement according to one embodiment of the invention,

FIGS. 2 and 2A show an electrode line according to one embodiment of the invention, with insertion instruments, and

FIGS. 3A to 3D show schematic diagrams to illustrate an aspect of the embodiment of the invention.

### DETAILED DESCRIPTION OF THE INVENTION

The following exemplary embodiment shows an application that will be important in the near future, placement of an endocardial electrode for left-ventricular stimulation in cardiac resynchronization therapy.

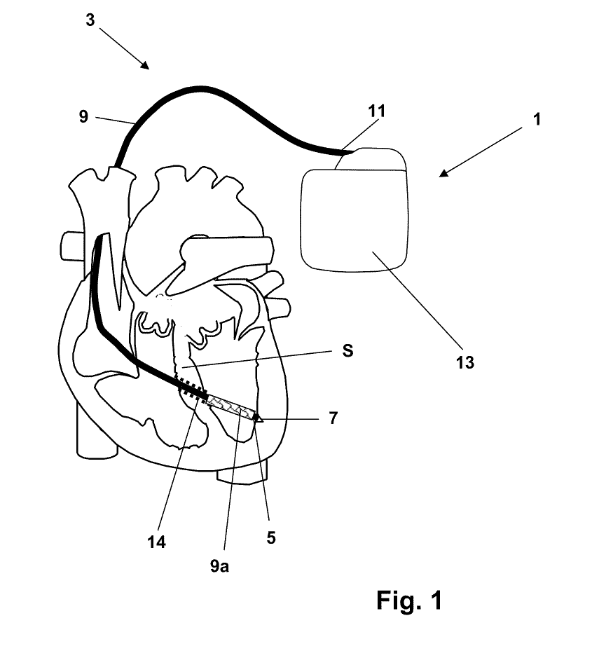

FIG. 1 shows a stimulation arrangement 1 with a stimulation and detection electrode line 3 permanently implanted in the left ventricle. The line 3 has an electric pole 5 for stimulation of the left-ventricular myocardium, fixation means 7 (screw, tines) in the distal area and a flexible line body 9 connected to a plug 11 for connection to an electronic implant 13, e.g., a cardiac pacemaker. The transseptal electrode shown here optionally has a fixation/closure mechanism 14 in the area of septum S. According to embodiments of the invention, a section 9a of the flexible line body 9 which is situated in the left ventricle is provided with an SiC coating 15 that promotes the formation of endogenous tissue and at the same time has antithrombotic properties.

A left-atrial application is likewise possible; in this case the electrode is placed in the left atrium by passing it through the atrial septum.

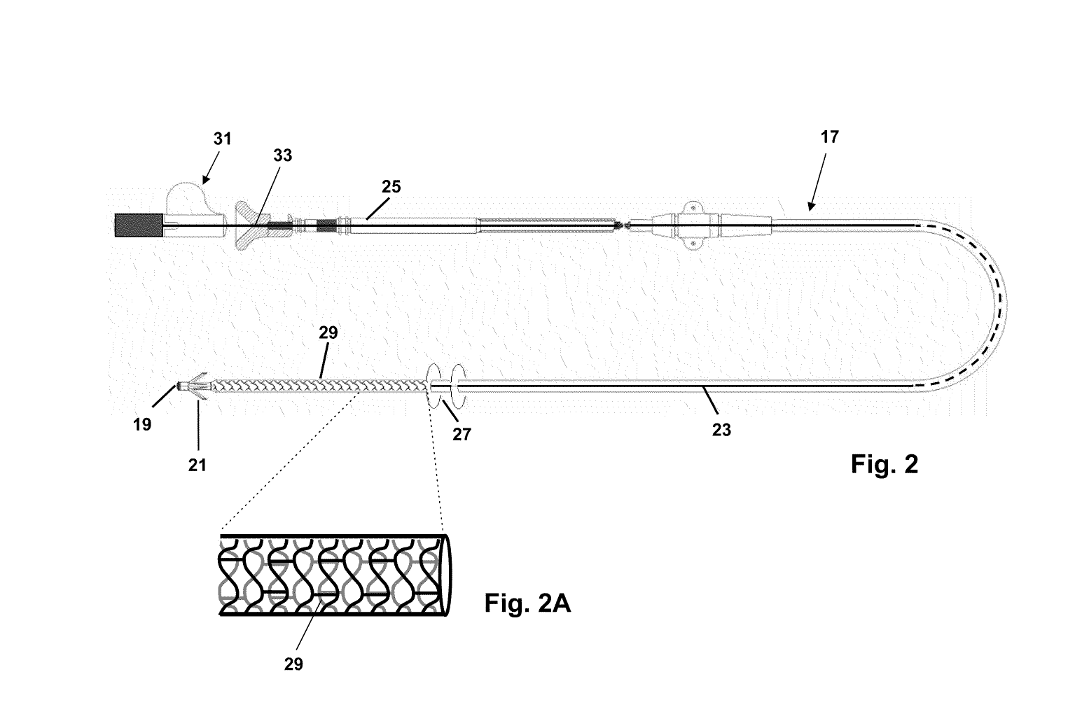

FIG. 2 shows as an embodiment a left-ventricular probe 17 with a unipolar stimulation pole 19, silicone tines 21 for fixation of the electrode in the myocardium, a flexible line body 23 and a connecting plug 25 corresponding to the IS-1 standard. Additionally, the probe 17 has a closure element 27 which, in the implanted state, is in the area of the ventricular septum. To stimulate rapid ingrowth of the electrode line in the left ventricle, the electrode body is covered with a metallic mesh 29 (preferably MP35N or cobalt-chromium) in the area of the left ventricle and/or this mesh is applied in an embedded carrier material or is partially or entirely embedded therein. The mesh 29 is coated with silicon carbide (SiC), which may also have amorphous structures (amorphous silicon carbide=a:SiC).

Instead of a mesh, a helical structure may also be used.

The figure additionally shows an insertion instrument 31, which serves to implant the electrode line in the left ventricle of the heart with the guidance of a guide wire 33. It is self-evident that during the implantation process, the closure element 27 is applied to the line body by a suitable mechanism (not part of embodiments of the invention) to allow it to pass with a low resistance through the corresponding vessels and the ventricular septum.

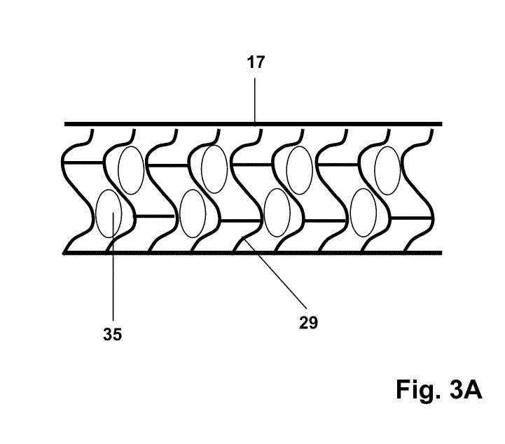

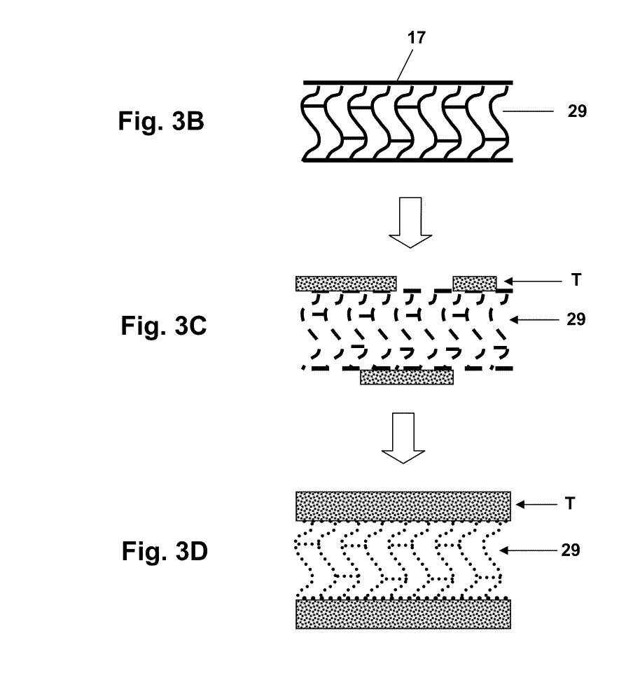

FIG. 3A shows in detail the SiC-coated mesh 29 applied in the area of the left ventricle in the probe and/or line 17. In this application, suitable active ingredients for promoting the ingrowth and/or active ingredients for inhibiting platelet aggregation, embedded in a biodegradable carrier material 35, are additionally introduced into the line body 23 in this area. The elution rate is selected so that the medication is released for a period of four to six weeks. This prevents thrombi from developing until conclusion of completely enclosing the electrode with endogenous tissue. The aforementioned active ingredients may also be introduced in a layer situated between the line body and the surface coating or surface structure. In this case, active ingredient depots or cavities filled with active ingredient are introduced in this layer, for example, ensuring that the medication will be released for a period of four to six weeks.

In another alternative, the mesh 29 may be completely embedded in a biodegradable carrier material designed like the abovementioned layer. In this case, the carrier material degrades, eluting the active ingredient(s) and at the same time releasing the surface structure or surface coating.

FIGS. 3B to 3D show in the form of diagrams the effect of a biodegradable material for the mesh 29 which promotes epithelialization. This mesh is made here of magnesium or a magnesium alloy (as in the so-called AMS stent of alloy WE 43, for example). With increasing ingrowth of body tissue T, the degradation of the magnesium compound takes place, so that after complete ingrowth (after approximately four to six weeks), the mesh structure has largely dissolved. It is self-evident that even with such a surface structure or surface coating of magnesium or magnesium alloy, suitable active ingredients for promoting the ingrowth and/or active ingredients for inhibiting platelet aggregation may be introduced into a biodegradable carrier 35 or an additional layer (as explained with regard to FIG. 3A). In this case, the layer may also be manufactured from a biodegradable matrix, e.g., poly-L-lactide.

In another embodiment, the surface structure or surface coating and the medication depot may be combined. In this case, the surface structure or surface coating consists of a biodegradable polymer, e.g., poly-L-lactide (PLLA) in which suitable active ingredients for promoting the ingrowth and/or active ingredients for inhibiting platelet aggregation are embedded or incorporated. In addition, a further biodegradable layer may also be provided with the same or additional active ingredients here.

The embodiment of the invention for left-ventricular/left-atrial sensors or catheters for left-ventricular/left-atrial medication administration corresponds to the aforementioned exemplary embodiment. Moreover, this embodiment of the invention is not limited to the examples explained above and the aspects emphasized above but is also possible in a variety of modifications which are within the scope of technical expertise.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. An implantable line for implantation in a blood vessel in or on a heart, or in a left ventricle or left atrium of the heart with perforation of an atrial or ventricular septum, comprising:
a line body that is elongated and flexible;
an electrode and/or a sensor and/or a medication-dispensing device in a distal segment of the line body; and,
wherein a part of the line body is situated in the blood vessel in or on the heart and is at least partially provided with a surface coating or surface structure that promotes ingrowth with endogenous tissue.

2. The implantable line according to claim 1, wherein the surface coating or surface structure that promotes ingrowth is provided in an area of the line body which is situated near a wall of the blood vessel in or on the heart.

3. The implantable line according to claim 1, wherein the surface structure or surface coating comprises an absorbable material, or magnesium or a magnesium alloy or a biodegradable polymer.

4. The implantable line according to claim 3, wherein the biodegradable polymer contains
an active ingredient that promotes the ingrowth
or additionally or alternatively contains
an active ingredient that inhibits platelet aggregation until complete enclosure with the endogenous tissue.

5. The implantable line according to claim 1, wherein the surface structure has a mechanical macrostructure, or a helical structure or a mesh-type structure or a stent-type structure.

6. The implantable line according to claim 1, wherein the surface coating has a geometric microstructure having structural dimensions in a range between 1 and 500 μm.

7. The implantable line according to claim 1, wherein the surface coating has a geometric microstructure having structural dimensions in a range between 5 and 200 μm.

8. The implantable line according to claim 1, wherein the surface coating has a geometric microstructure having structural dimensions in a range between 10 and 50 μm.

9. The implantable line according to claim 1, wherein the surface coating or the surface structure is applied to a carrier material embedded in the line body, wherein the carrier material is a continuous layer between the line body and the surface coating or the surface structure.

10. The implantable line according to claim 9, wherein the carrier material contains active ingredients to promote the ingrowth in a depot storage form.

11. The implantable line according to claim 9, wherein the carrier material alternatively or additionally has active ingredients that inhibit platelet aggregation until complete enclosure with the endogenous tissue.

12. The implantable line according to claim 1, further comprising a fixation means provided on or near a distal end of the line body for fixation in a wall section in or on the blood vessel in or on the heart.

13. The implantable line according to claim 1, further comprising multiple sensing electrodes and/or stimulation electrodes and/or shock electrodes.

14. The implantable line according to claim 13, wherein at least a portion of the multiple sensing electrodes and/or stimulation electrodes and/or shock electrodes is provided with the surface coating or surface structure that promotes ingrowth, in a stent-type or mesh-type or helical structure.

15. The implantable line according to claim 1, wherein the part of the line body, which is in or on the blood vessel in or on the heart has a preshaped curvature, in particular in a J, S or U shape, designed such that the preshaped curvature is eliminated on insertion of a guide wire and is restored after removal of the guide wire and/or under influence of body heat.

16. The implantable line according to claim 15, wherein a position of an area provided with the surface coating or surface structure on the preshaped curvature of the line body is configured to the preshaped curvature, such that a section of the line body carries the surface coating or surface structure, which is in contact with a wall of the blood vessel in or on the heart.
